(19) United States
(12) Patent Application Publication
(10) Pub. No.: US 2020/0324064 A1
(43) Pub. Date: Oct. 15, 2020

(54) Inhalation Device for Administering Powdered Pharmaceutical Composition

(71) Applicants: Cai Gu Huang, Shrewsbury, MA (US); Jian Cai, Shanghai (CN)

(72) Inventors: Cai Gu Huang, Shrewsbury, MA (US); Jian Cai, Shanghai (CN)

(73) Assignee: Cai Gu Huang, Shrewsbury, MA (US)

(21) Appl. No.: 16/837,499

(22) Filed: Apr. 1, 2020

Related U.S. Application Data
(60) Provisional application No. 62/831,719, filed on Apr. 10, 2019.

(51) Int. Cl. A61M 15/00

(52) U.S. Cl. CPC A61M 15/0051; A61M 15/0073; A61M 2202/064

(57) ABSTRACT

This invention relates to an inhalation device by which a user can inhale powdered pharmaceutical compositions or medications. Powdered medication is held in blister packs containing at least one or two separated pockets for two or more active pharmaceutical ingredients and is inhaled by users through inhalation devices. The invention also relates to an inhalation device for powdered pharmaceutical compositions comprising a medication blister pack containing two separated pockets to hold two active pharmaceutical ingredients, an opening station, and an outlet, the blister pack comprising two pockets for powdered medication contained between two members peelably secured to one another.

### PRIORITY STATEMENT

This application claims the benefit of the filing date of U.S. Provisional Patent Application No. 62/831,719, filed on Apr. 10, 2019, which is incorporated herein by reference in its entirety.

### FIELD AND BACKGROUND OF THE INVENTION

This invention refers to an inhalation device by which a user can inhale powdered pharmaceutical compositions or medications. Powdered medication is held in blister packs containing at least one or two separated pockets for two or more active pharmaceutical ingredients and is inhaled by users through inhalation devices. This inhalation device includes a puncturing device or a peeling device to puncture or open each blister in turn for inhalation. In an embodiment, the present invention provides an inhalation device comprising blister packs, which is of a convenient size to hold in a user's hand but large enough to process a medication blister pack with a large number of discrete unit doses.

### SUMMARY OF THE INVENTION

The present invention provides an inhalation device for powdered pharmaceutical compositions provided in a medication blister pack. In an embodiment, the medication blister pack comprises an elongated base sheet having a first end, a second end, and multiple recesses between the first end and the second end; an elongated lid sheet hermetically sealed to the base sheet so that the base sheet and lid sheet can be peeled apart. The multiple recesses comprise at least a first pocket for powdered medication and a second pocket for powdered medication. At least one pocket for powdered medication is defined between the base sheet and the lid sheet, peelably secured to one another. The first pocket comprises at least a first active pharmaceutical ingredient, and the second pocket comprises at least a second active pharmaceutical ingredient, which may be the same or different. The inhalation device comprises an opening station connected with at least one pocket. The inhalation device comprises means for peeling the members apart at the opening station so that the pocket is opened. The device comprises at least one outlet, which is open to and communicates with the open pocket. A user can inhale the powdered medication from this open pocket through the outlet.

The present invention provides a medication blister pack for the inhalation device. This medication blister pack comprises at least one or two separated pockets holding two or more active pharmaceutical ingredients, with each pocket containing a single active pharmaceutical ingredient or a combination of active pharmaceutical ingredients. The medication blister pack in the inhalation device may be formed from by two elongated sheets. One of the elongated sheets of the medication blister pack is a base sheet which has multiple recesses along the sheet. The other is a lid sheet. The lid sheet is sealed to the base sheet hermetically, but the two sheets can be peeled apart. These sheets define multiple pockets, which may be of any suitable shape, such as, for example, oval or round. Inhalable powdered medications are located in the pockets. In an embodiment, these sheets are sufficiently flexible to be wound into a roll. The medication blister pack may include a means for indexing the pockets or doses during operation of the inhalation device.

In an embodiment of the present invention, one pocket of the medication blister pack will hold a first active pharmaceutical ingredient, optionally in a pharmaceutically acceptable composition suitable for administration by inhalation, and a second pocket of the medication blister pack will hold a second active pharmaceutical ingredient, optionally in a pharmaceutical composition suitable for administration by inhalation.

The present invention further provides a medication blister pack comprising at least a first pocket for powdered medication, a second pocket for powdered medication, a first active pharmaceutical ingredient, and a second active pharmaceutical ingredient. In an embodiment of the current invention, the medication blister pack is designed to accommodate at least two or more powdered pharmaceutical compositions, A and B. Each of compositions A and B comprises one or more active ingredients formulated in a powdered pharmaceutically acceptable composition. The compositions A and B are filled into the pockets in arrangements such as those described in FIG. 5.

In an embodiment, the two different powdered pharmaceutical compositions in multiple unit doses are provided in an alternating A-B-A-B-A-B . . . (FIG. 5b) sequence. In another embodiment, two pockets containing different pharmaceutical compositions A and B are arranged next to each other, approximately equidistant from the end of the medication blister pack (FIG. 5c). In another embodiment, different pharmaceutical compositions A and B are placed into two adjoining pockets which have a separation or barrier wall between them to separate the powdered pharmaceutical compositions A and B (FIG. 5d). The powdered pharmaceutical compositions are formulated into the composition A representing by the dark oval shape pocket and composition B representing the blank oval pocket as described in FIGS. 5b, 5c and 5d.

In an embodiment, the current invention provides a medication blister pack comprising at least one or two separated pockets holding two or more active pharmaceutical ingredients, in which the two different powdered pharmaceutical compositions in multiple unit doses are present in an alternating A-B-A-B-A-B . . . (FIG. 5b) sequence, wherein the powdered pharmaceutical composition A comprises fluticasone furoate as the active ingredient, and the powdered pharmaceutical composition B comprises vilanterol trifenatate as the active ingredient.

In an embodiment, the current invention provides a medication blister pack comprising two separated pockets holding two or more active pharmaceutical ingredients, in which the two different powdered pharmaceutical compositions in multiple unit doses are present in an alternating A-B-A-B-A-B . . . (FIG. 5b) sequence, wherein the powdered pharmaceutical composition A comprises umeclidinium or its salts as the active ingredient, and the powdered pharmaceutical composition B comprises vilanterol trifenatate as the active ingredient.

In an embodiment, the current invention provides a medication blister pack comprising at least one or two separated pockets holding two or more active pharmaceutical ingredients, in which the two different powdered pharmaceutical compositions in multiple unit doses are present in an alternating A-B-A-B-A-B . . . (FIG. 5b) sequence, wherein the powdered pharmaceutical composition A comprises umeclidinium or its salts as the active ingredient, and the powdered pharmaceutical composition B comprises vilanterol trifenatate and fluticasone furoate as the active ingredients.

In an embodiment, the current invention provides a medication blister pack comprising two separated pockets holding two active pharmaceutical ingredients, in which the two different powdered pharmaceutical compositions in multiple unit doses are present in an alternating A-B-A-B-A-B . . . (FIG. 5b) sequence, wherein the powdered pharmaceutical composition A comprises glycopyrrolate bromide as the active ingredient, and the powdered pharmaceutical composition B comprises indacaterol maleate as the active ingredient.

In an embodiment, the current invention provides a medication blister pack comprising two separated pockets holding two active pharmaceutical ingredients, in which the two different powdered pharmaceutical compositions in multiple unit doses are present in an alternating A-B-A-B-A-B . . . (FIG. 5b) sequence, wherein the powdered pharmaceutical composition A comprises aclidinium bromide as the active ingredient, and the powdered pharmaceutical composition B comprises formoterol fumarate as the active ingredient.

In an embodiment, the current invention provides a medication blister pack comprising two separated pockets holding two active pharmaceutical ingredients, in which the two different powdered pharmaceutical compositions in multiple unit doses are present in two pockets containing different pharmaceutical compositions A and B, arranged next to each other and approximately equidistant from the end of the medication blister pack (FIG. 5c), wherein the powdered pharmaceutical composition A comprises indacaterol maleate as the active ingredient, and the powdered pharmaceutical composition B comprises glycopyrrolate bromide as the active ingredient.

In an embodiment, the current invention provides a medication blister pack comprising two separated pockets holding two active pharmaceutical ingredients, in which the two different powdered pharmaceutical compositions in multiple unit doses are present in two pockets containing different pharmaceutical compositions A and B, arranged next to each other and approximately equidistant from the end of the medication blister pack (FIG. 5c), wherein the powdered pharmaceutical composition A comprises aclidinium bromide as the active ingredient, and the powdered pharmaceutical composition B comprises formoterol fumarate as the active ingredient.

In an embodiment, the current invention provides a medication blister pack comprising two separated pockets holding two active pharmaceutical ingredients, in which the two different powdered pharmaceutical compositions in multiple unit doses are present in two pockets containing different pharmaceutical compositions A and B, arranged next to each other and approximately equidistant from the end of the medication blister pack (FIG. 5c), wherein the powdered pharmaceutical composition A comprises umeclidinium or its salts as the active ingredient, and the powdered pharmaceutical composition B comprises vilanterol trifenatate as the active ingredient.

In an embodiment, the current invention provides a medication blister pack comprising two separated pockets holding two active pharmaceutical ingredients, in which the two different powdered pharmaceutical compositions in multiple unit doses are present in two pockets containing different pharmaceutical compositions A and B, arranged next to each other and approximately equidistant from the end of the medication blister pack (FIG. 5c), wherein the powdered pharmaceutical composition A comprises umeclidinium or its salts as the active ingredient, and the powdered pharmaceutical composition B comprises fluticasone furoate as the active ingredient.

In an embodiment, the current invention provides a medication blister pack comprising at least one or two separated pockets holding two or more active pharmaceutical ingredients, in which the two different powdered pharmaceutical compositions in multiple unit doses are present in two pockets containing different pharmaceutical compositions A and B, arranged next to each other and approximately equidistant from the end of the medication blister pack (FIG. 5c), wherein the powdered pharmaceutical composition A comprises umeclidinium or its salts as the active ingredient, and the powdered pharmaceutical composition B comprises vilanterol trifenatate and fluticasone furoate as the active ingredients.

In an embodiment, the current invention provides a medication blister pack comprising two separated pockets holding two active pharmaceutical ingredients, in which the two different powdered pharmaceutical compositions in multiple unit doses are present in two pockets containing different pharmaceutical compositions A and B, arranged next to each other and approximately equidistant from the end of the medication blister pack (FIG. 5c), wherein the powdered pharmaceutical composition A comprises fluticasone furoate as the active ingredient, and the powdered pharmaceutical composition B comprises vilanterol trifenatate and umeclidinium or its salts as the active ingredients.

In an embodiment, the current invention provides a medication blister pack comprising two separated pockets holding two active pharmaceutical ingredients, in which the two different powdered pharmaceutical compositions in multiple unit doses are present in two adjoining pockets with a separation wall between them to separate the powdered pharmaceutical compositions A and B (FIG. 5d), wherein the powdered pharmaceutical composition A comprises indacaterol maleate as the active ingredient, and the powdered pharmaceutical composition B comprises glycopyrrolate bromide as the active ingredient.

In an embodiment, the current invention provides a medication blister pack comprising two separated pockets holding two active pharmaceutical ingredients, in which the two different powdered pharmaceutical compositions in multiple unit doses are present in two adjoining pockets with a separation wall between them to separate the powdered pharmaceutical compositions A and B (FIG. 5d), wherein the powdered pharmaceutical composition A comprises aclidinium bromide as the active ingredient, and the powdered pharmaceutical composition B comprises formoterol fumarate as the active ingredient.

In an embodiment, the current invention provides a medication blister pack comprising two separated pockets holding two active pharmaceutical ingredients, in which the two different powdered pharmaceutical compositions in multiple unit doses are present in two adjoining pockets with a separation wall between them to separate the powdered pharmaceutical compositions A and B (FIG. 5d), wherein the powdered pharmaceutical composition A comprises umeclidinium or its salts as the active ingredient, and the powdered pharmaceutical composition B comprises vilanterol trifenatate as the active ingredient.

In an embodiment, the current invention provides a medication blister pack comprising at least one or two separated pockets holding two active pharmaceutical ingredients, in which the two different powdered pharmaceutical compositions in multiple unit doses are present in two adjoining pockets with a separation wall between them to separate the powdered pharmaceutical compositions A and B (FIG. 5d), wherein the powdered pharmaceutical composition A comprises umeclidinium or its salts as the active ingredient, and the powdered pharmaceutical composition B comprises fluticasone furoate as the active ingredient.

In an embodiment, the current invention provides a medication blister pack comprising at least one or two separated pockets holding two active pharmaceutical ingredients, in which the two different powdered pharmaceutical compositions in multiple unit doses are present in two adjoining pockets with a separation wall between them to separate the powdered pharmaceutical compositions A and B (FIG. 5d), wherein the powdered pharmaceutical composition A comprises umeclidinium or its salts as the active ingredient, and the powdered pharmaceutical composition B comprises vilanterol trifenatate and fluticasone furoate as the active ingredients.

In an embodiment, the current invention provides a medication blister pack comprising at least one or two separated pockets holding two active pharmaceutical ingredients, in which the two different powdered pharmaceutical compositions in multiple unit doses are present in two adjoining pockets with a separation wall between them to separate the powdered pharmaceutical compositions A and B (FIG. 5d), wherein the powdered pharmaceutical composition A comprises fluticasone furoate as the active ingredient, and the powdered pharmaceutical composition B comprises vilanterol trifenatate and umeclidinium or its salts as the active ingredients.

In an embodiment, the current invention provides a medication blister pack comprising at least two separated pockets holding two or more active pharmaceutical ingredients selected from the group consisting of aclidinium bromide, formoterol fumarate, umeclidinium and pharmaceutically acceptable salts thereof, vilanterol trifenatate, indacaterol maleate, fluticasone furoate and glycopyrrolate bromide or any combinations of any two or three active pharmaceutical ingredients.

Preferably, the current invention provides a medication blister pack comprising two separated pockets holding two active pharmaceutical ingredients comprising vilanterol trifenatate and formoterol fumarate.

In an embodiment, the current invention provides a medication blister pack comprising two separated pockets holding two active pharmaceutical ingredients comprising vilanterol trifenatate and umeclidinium bromide.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

FIGS. 1 to 4A show an embodiment of the invention. FIG. 1 is an under plan view; FIG. 2 is a section on line A-A in FIG. 1; FIG. 3 is a section on line B-B in FIG. 1; and FIG. 4A is an exploded view on a smaller scale.

FIGS. 4B to 4E show an embodiment in successive stages of operation, and FIG. 4F is a section taken on line A-A in FIG. 4B.

FIG. 5 is a perspective view showing flexible blister strips of various embodiments of the invention.

FIGS. 6 to 10 show a modified clutch which may be used in those embodiments of the invention which require it, and are, respectively, a front view, a top view, a back view, a left side view and an axonometric view.

FIG. 11 is an exploded perspective view showing an embodiment of clutch.

FIG. 12A is an exploded perspective view of yet another embodiment of clutch.

FIG. 12B is transverse section through the clutch shown in FIG. 12A.

### DETAILED DESCRIPTION OF THE INVENTION

The present invention provides an inhalation device for one or more powdered pharmaceutical compositions provided in an medication blister pack. For purposes of describing the invention, reference now will be made in detail to embodiments and/or methods of the invention, one or more examples of which are illustrated in or with the drawings. Each example is provided by way of explanation of the invention, not limitation of the invention. In fact, it will be apparent to those skilled in the art that various modifications and variations can be made in the present invention without departing from the scope or spirit of the invention. For instance, features or steps illustrated or described as part of one embodiment, can be used with another embodiment or steps to yield a still further embodiments or methods. Thus, it is intended that the present invention covers such modifications and variations as come within the scope of the appended claims and their equivalents.

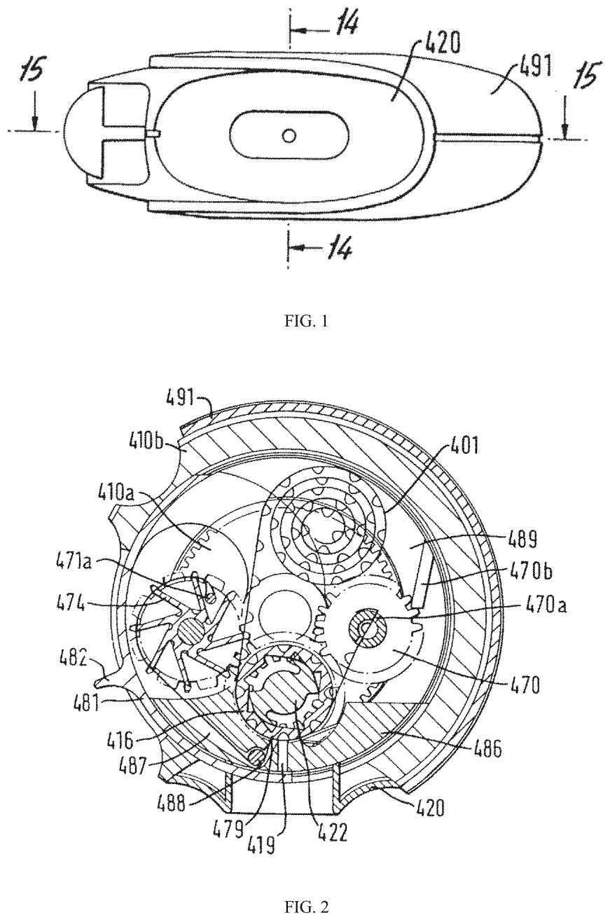

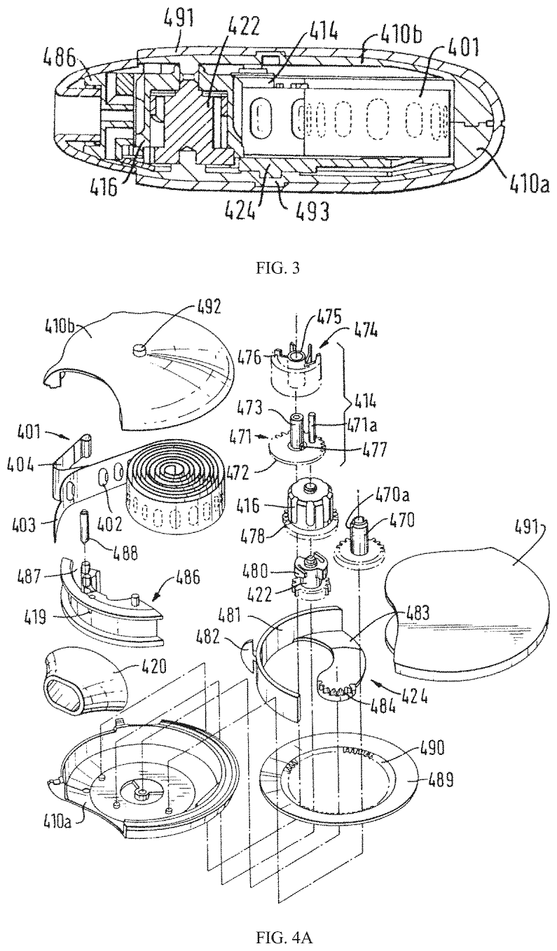

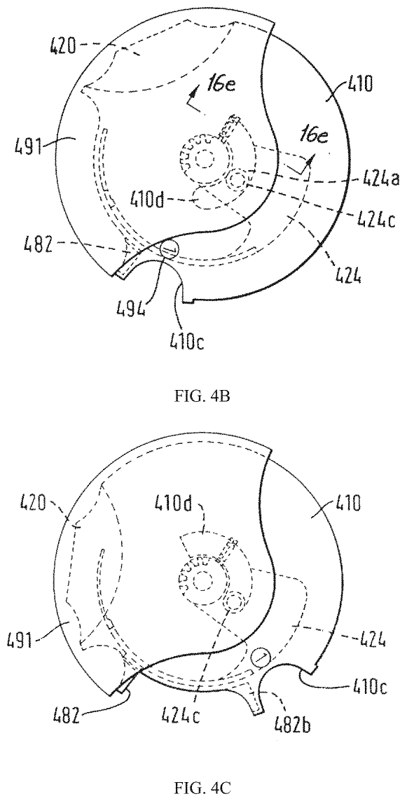

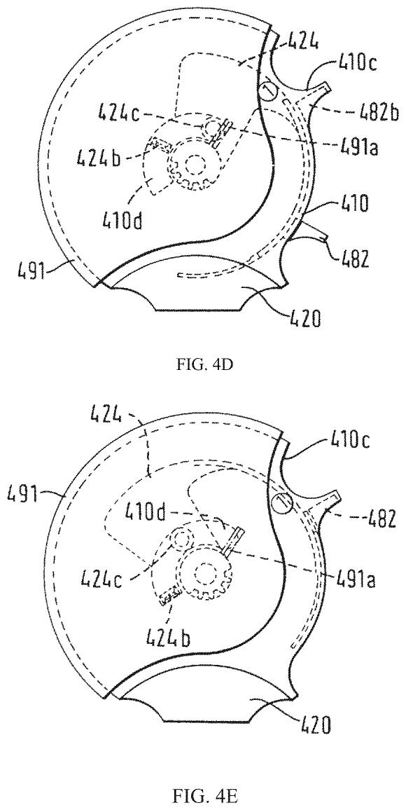

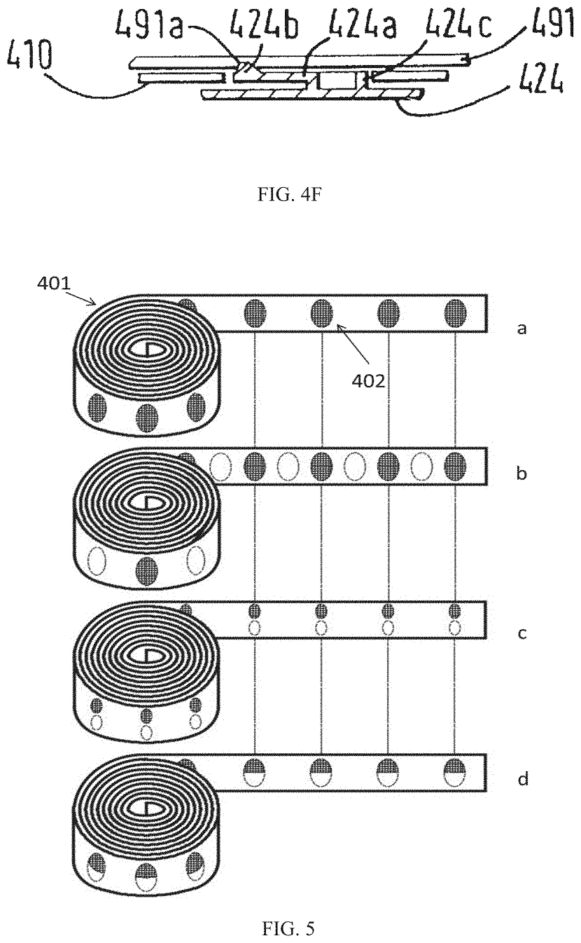

FIGS. 1, 2, 3 and 4A show an embodiment of the invention. The device receives a flexible blister strip, here denoted as 401, comprising a base sheet 403 in which pockets 402 are defined and a lid sheet 404. The blister strip 401, is shown most clearly in FIG. 4A. The lid sheet 404 has a loop 404a formed at the leading end thereof for engagement over a post 471a extending upwardly from a toothed wheel 471 (described below). The base sheet has a lead portion 403a of reduced width for engagement in a slot 470a formed in the base winding wheel 470 (described below). The leading end portions of the base sheet and lid sheet are not sealed together, as can be seen in FIG. 4A.

The body 410 comprises a base 410a and a top 410b both of generally circular shape. When the device is assembled the base and top are snap-fitted together. The body defines a single internal chamber within which the strip 401 is housed and within which are also housed a wheel 414 for winding up the used portion of the lid sheet 404, a base winding wheel 470 and an index wheel 416. The index wheel 416 is hollow and an index ratchet wheel 422 is housed within it. All the wheels just mentioned are mounted in the chamber defined by the body, for rotational movement with respect thereto. A pawl 470b is attached to the body 410 and engages the teeth of the base winding wheel 470 to prevent the wheel moving counterclockwise, thus ensuring that the strip 401 can only proceed forwards through the device.

The lid winding wheel 414 is formed in two parts, namely a toothed wheel 471 having teeth 472 and a shaft 473, and a collapsible wheel 474 having a hollow central shaft 475 and a plurality of resilient arms 476, for example, as shown, eight such arms, extending from the central shaft 475 each at an angle to a radius. The toothed wheel 471 has a lug 477 which engages in a corresponding notch in the shaft 475 so that the wheels 471 and 474 rotate in unison.

The hollow index wheel 416 has external teeth 478 which mesh with the teeth of the base winding wheel 470 and the teeth of the wheel 471. Ratchet teeth 479 are formed on the internal walls of the index wheel 416, and the index ratchet wheel 422 has two pawls 480 which engage the ratchet teeth 479.

The device further comprises a lever 424 which comprises an arcuate wall 481 with a finger tab 482, and an arm 483 which extends inwardly from the wall 481 and carries an arcuate array of teeth 484 at its distal end. The lever is pivotally mounted to the center of the base 410a for movement about an axis which is at the center of the pitch circle of the teeth 484, the teeth 484 mesh with the teeth 485 on the index ratchet wheel 422.

A manifold 486 provides communication between the chamber within the body 410 and a mouthpiece 420. The manifold has a powder outlet 419 and also has a passageway 487 to allow used lid strip 404 to pass to the collapsible wheel 474. Optionally, a roller 488 may be provided to guide the strip 404 into the passageway 487.

A dose monitor ring 489 having teeth 490 is arranged to be rotatable within the body base 410a. On its lower surface this bears indicia (not visible in the drawings) which can be viewed by the user through a window 494 in the body 410. It will be noted from FIGS. 4B to 4E that the window can be seen both when the cover 491 (see below) is closed and when it is open. The indicia indicate either exactly or approximately the number of doses left (or the number of doses used, if preferred). The ring 489 is rotated by virtue of the fact that its teeth 490 are engaged by the teeth 478 of the index wheel.

The device is provided under a cover 491 which is pivotally mounted on the body 410 by means of a lug 492 on the body top 410b and a corresponding lug 493 on the body base 410a. The cover is pivotal between an open position (shown in FIG. 2) in which the mouthpiece is exposed and a closed position in which it is not, as is described more fully below.

In operation, the user moves the cover 491 to its open position and then presses on the finger tab 482 of the lever 424 to cause it to move as the lever pivots. This makes the index ratchet wheel 422 rotate which, via the pawls 480, causes the index wheel 416 also to rotate. Rotation of the index wheel 416 produces rotation of both the base winding wheel 470 and the lid winding wheel 414, thus peeling the base sheet and lid sheet apart over a distance sufficient to expose a previously unopened pocket 402 opposite the end of the powder outlet 419 in the manifold 486. The user can then inhale through the mouthpiece.

Successive stages in the operation of the device are shown in FIG. 4B to 4E. The device is in its closed position in FIG. 4B. The finger tab 482 of the lever 424 is at this stage in a recess 482b formed in the body 410 (seen more clearly in FIGS. 4C and 4D). The cover 419 is held stationary as the body 410 is rotated counterclockwise, a recess 410c being provided in the periphery of the body to enable the user to insert a finger for this purpose. The device is thus moved to the partly open position shown in FIG. 4C. During this process the lever 424 remains stationary with respect to the cover 491.

This is achieved by the lever being provided internally with a resilient arm 424a the tip 424b of which engages in a recess 491a in the cover 491. The arm 424a is attached to the lever 424 via a cylindrical member 424c. As viewed in FIG. 4B, the arm 424a extends counterclockwise from the member 424c over an arc of about 90°. The cylindrical member 424c is guided in an arcuate slot 410d formed in the body 410. The slot 410d extends through an arc of about 180°, and in FIG. 4B the member 424c is shown as being approximately half way along its length. In FIG. 4C it is shown as being at one end.

The user continues to rotate the body 410 from the position shown in FIG. 4C to the position shown in FIG. 4D. During this further rotation tip 424b of the arm 424a jumps out of the recess 491a. This occurs because, with the member 424c at one end of the slot 410d, movement of the body 410 carries the member 424c with it in a counterclockwise direction and hence compels the arm 424a likewise to move counterclockwise. The user then moves the lever 424 by pushing on the finger tab 482 to cause it to rotate counterclockwise through the position shown in FIG. 4D to the position shown in FIG. 4E where the finger tab 482 re-enters the recess 482b. The steps thus far described both expose the mouthpiece 420 and open a fresh blister. The device is therefore now ready for the user to inhale.

After use, the body 410 is rotated clockwise, the lever 424 moving in unison with the body, to bring the device back to the position of FIG. 4B.

It will be noted that the collapsible wheel 474 in effect assumes the function of the clutch. As more lid sheet is wound onto the wheel 474 the arms 476 gradually flex inwardly, and the effect is to keep the external diameter of the reel of wound up lid sheet substantially constant, while the internal diameter thereof gradually decreased.

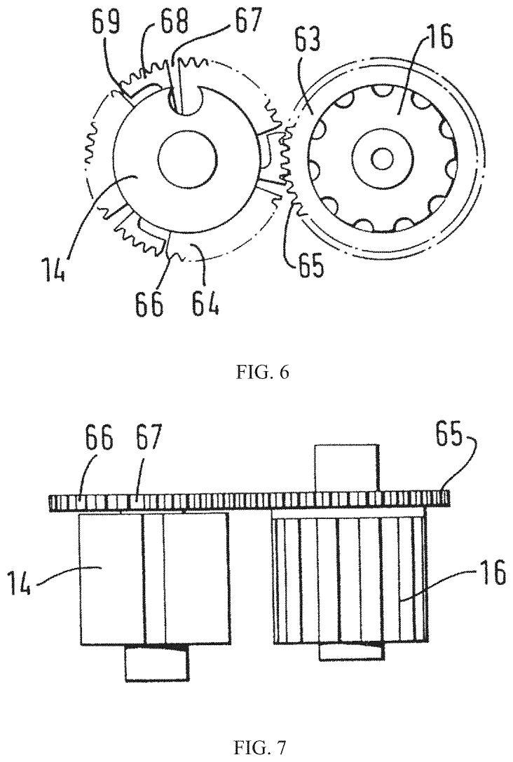

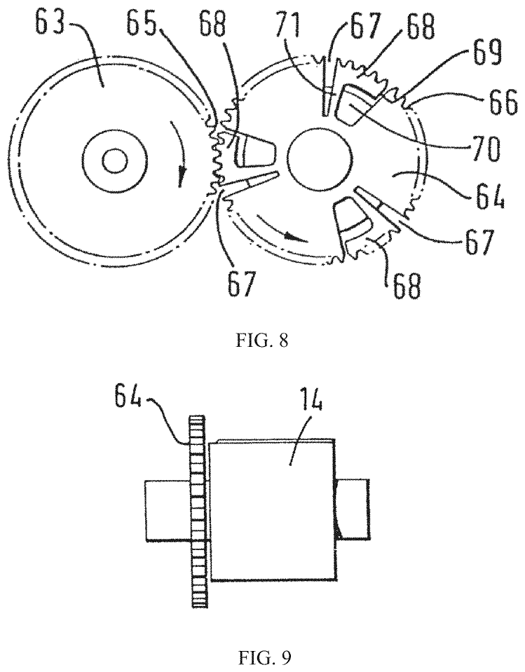

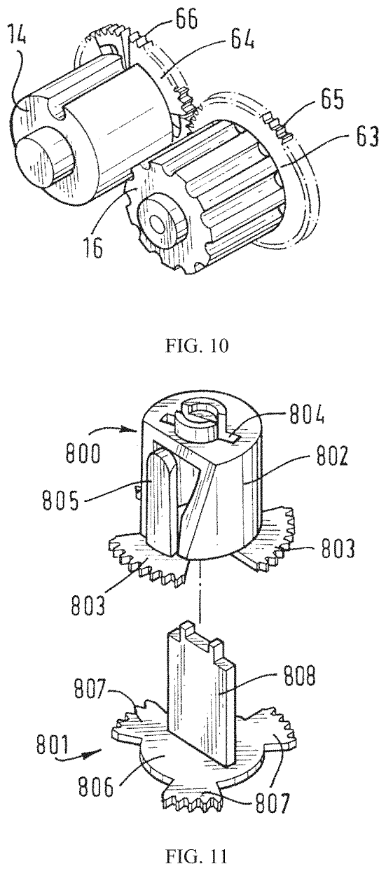

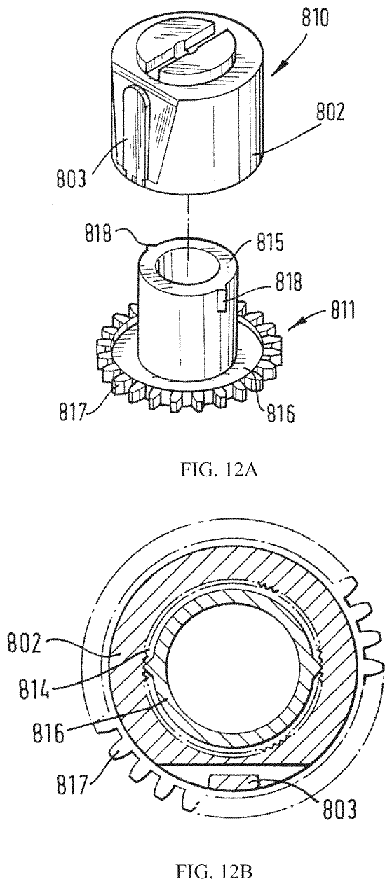

Instead of the wheel 414 with its collapsible wheel 474 it is possible to use the alternative structure shown in FIG. 11 or that shown in FIGS. 12A and 12B. The principle of operation of the structure shown in FIG. 11 is very similar to that of the clutch arrangement shown in FIGS. 6 to 10. The structure of FIG. 11 comprises two components 800 and 801. The component 800 comprises a generally cylindrical hollow housing 802 open at its lower end and three arcuate arrays of teeth 803. The cylinder 802 has a slot 804 extending through the upper surface thereof, and a post 805 for receiving the leading end of the lid sheet. The component 801 comprises a disc 806 provided with three arcuate arrays of teeth 807, and an upright member 808 extending upwardly from the disc 806. The member 808 is formed of a material, example a plastics material, which is resilient in torsion.

The two components 800 and 801 are snap-fitted together so that the upper end of the member 808 is received in the slot 804 and cannot rotate with respect thereto. The arrays of teeth 803 and 807 are coplanar and alternate with one another. The teeth 803 and 807 mesh with the teeth 478 of the index wheel. Each array 807 is separated from one of the adjacent arrays 803 (but not from the other) by a gap equal to one tooth. Thus, there are three gaps, each of one tooth width, around the assembled arrays. Because the member 808 can flex in torsion, the disc 806 is free to move back and forth between a position in which the gaps are each on one side of a respective array 807 and a position in which the gaps are each on the other side of a respective array 807. This has the effect of producing slippage of the structure shown in FIG. 11 with respect to the index wheel.

The structure shown in FIG. 12A is a slipping clutch. It comprises two components 810 and 811, snap-fitted together. The component 810 comprises a generally cylindrical housing 812 open at its lower end and having a post 813 for receiving the leading end of the lid sheet. The interior of the housing 812 is provided with longitudinally extending serrations 814, as can be seen in FIG. 12B. The component 811 comprises a cylinder 815 which extends upwardly from a disc 816 provided with teeth 817. The teeth 817 mesh with the teeth 478 of the index wheel. The cylinder 815 is provided on its outer surface with a pair of pips 818 which are in interfering engagement with the serrations 814. When the rotational force applied by the component 811 to the component 810 is below a predetermined level the components rotate together. However, the cylinder is made of a material, for example a plastics material, which can deform radially, and when the rotational force exceeds the predetermined level such deformation takes place, permitting the pips 818 to move over the serrations 814.

Although in the embodiment of FIGS. 1 to 4A, with or without the modifications of FIGS. 11 and 12A, the base sheet is wound up as well as the lid sheet, it is not necessary for there also to be a slipping clutch or the like between the index wheel and the base winding wheel. The diameter of the base winding wheel is so chosen that initially the base sheet is wound up only very loosely, and the tightness with which the sheet is wound increases during operation but without ever reaching an unacceptable level. In theory, the base sheet could be wound up precisely via a slipping clutch or the like, with the lid sheet being only loosely wound, but in practice it is much easier to wind up the lid precisely because it is flat and because it is thinner than the base sheet.

The inhalation device shown in FIGS. 1 to 4A can achieve inhalable drug delivery. In an embodiment, the inhalation device is provided with a medication blister pack containing at least one or two separated pockets for two or more active pharmaceutical ingredients. A number of embodiments are provided below.

FIG. 5a shows strip 401 in which each pocket 402 contains the same medication. In this embodiment, for each use, a user can inhale the powdered medication in one pocket 402, and drug powder flows out of the device from the same powder outlet.

FIG. 5b shows another embodiment. In this embodiment, the space between each pocket decreases by half compared to FIG. 5a. In this embodiment, two types of drug powder are included in separate pockets, in which each pocket has a different drug powder from its neighboring pockets. Each time the finger tab is activated, lid sheet 404 will be peeled apart from base sheet 403, and two pockets will be exposed to the powder outlet. A user can then inhale two types of drugs at the same time. In this embodiment, the inhalation device may be designed with two outlets to accommodate the two pockets.

FIG. 5c shows a third embodiment. In this embodiment, the size and position of pockets 402 in strip 401 are modified. As shown in FIG. 5c, each pocket 402 is smaller than in FIG. 5a, and the pockets containing different types of drug powder are arranged next to each other, approximately equidistant from the end of the blister strip. Each time the finger tab is activated, two pockets 402 in one row will be peeled open. Thus, users can inhale two types of drug powder at the same time through one outlet. This embodiment reduces the difficulty of injection molding and manufacturing of the device, and the difficulty of assembly of the device.

FIG. 5d shows a fourth embodiment. Strip 401 in this embodiment is similar to the one in FIG. 5a. Each pocket of base sheet 403 includes two adjoining smaller pockets and a separation wall to separate two types of drug powder. In addition, this separation wall also strengthens the pocket and reduces the possibility of deformation of the pocket. The rest of this embodiment is analogous to the third embodiment. Users can inhale two types of drug powder from one pocket and one outlet at the same time.

In a fifth embodiment, the drug filled pockets 402 of strip 401 are spaced as in FIG. 5a but arranged alternately, as in FIG. 5b. As shown in FIG. 5b, each pocket is filled with different drug powder to its neighboring pocket. Each time the finger tab is activated, a user can inhale the drug powder from one pocket. To administer both drug products, the user will activate the finger tab and inhale twice in succession.

In the sixth embodiment, two types of drug powders are mixed physically, and then the combined drug powder is filled into each pocket. Thus each pocket has two types of active pharmaceutical ingredients. When the finger tab is activated, a user can inhale both types of drugs at the same time.

The seventh embodiment has the same device structure as the sixth embodiment. Two types of drug powders are recrystallized and then filled into each pocket. In this embodiment, users operate the device the same as in the sixth embodiment, and both active pharmaceutical ingredients are administered at the same time.

While various embodiments of the present invention have been described above, it should be understood that they have been presented by way of example only, and not limitation. For example, the present invention is not limited to the physical arrangements or dimensions illustrated or described. Nor is the present invention limited to any particular design or materials of construction. As such, the breadth and scope of the present invention should not be limited to any of the above-described exemplary embodiments, but should be defined only in accordance with the following claims and their equivalents.

What is claimed is:

1. An inhalation device for powdered pharmaceutical compositions comprising a medication blister pack comprising an elongated base sheet having a first end, a second end, and multiple recesses between the first end and the second end; an elongated lid sheet hermetically sealed to the base sheet so that the base sheet and lid sheet can be peeled apart; wherein the multiple recesses comprise a first pocket for powdered medication and a second pocket for powdered medication, with the pockets defined between the base sheet and the lid sheet; wherein the first pocket comprises a first active pharmaceutical ingredient, and the second pocket comprises a second active pharmaceutical ingredient.

2. The inhalation device according to claim 1, wherein the medication blister pack is a blister strip.

3. The inhalation device according to claim 2, wherein the blister strip is flexible.

4. The inhalation device according to claim 2, wherein the blister strip can be wound into a roll.

5. The inhalation device according to claim 1, wherein the first pocket and the second pocket of the medication blister pack are repeated in multiple unit doses in an alternating A-B-A-B-A-B sequence.

6. The inhalation device according to claim 1, wherein the first pocket and the second pocket of the medication blister pack are arranged next to each other approximately equidistant from the first end of the medication blister pack.

7. The inhalation device according to claim 1, wherein the first pocket and the second pocket of the medication blister pack are repeated in multiple unit doses in an alternating A-B-A-B-A-B sequence, or wherein the first pocket and the second pocket are arranged next to each other approximately equidistant from the first end of the medication blister pack.

8. The inhalation device according to claim 1, wherein the first pocket of the medication blister pack adjoins the second pocket with a separation wall to separate the first active pharmaceutical ingredient from the second active pharmaceutical ingredient.

9. The inhalation device according to claim 1, wherein the medication blister pack comprises a lead portion on the first end.

10. The inhalation device according to claim 1, which comprises a body comprising a base and a top, both of generally circular shape.

11. The inhalation device according to claim 1, comprising a slipping clutch.

12. The inhalation device according to claim 1, which comprises a body with a pawl attached thereto.

13. The inhalation device according to claim 1, which comprises a lever, comprising an arcuate wall with a finger tab, and an arm which extends inwardly from the wall.

14. The inhalation device according to claim 1, which comprises a body defining a single internal chamber.

15. The inhalation device according to claim 13, which comprises a body comprising a base and a top, wherein the lever is pivotally mounted to the base.

16. The inhalation device according to claim 1, comprising a roller to guide the medication blister pack into the device.

17. The inhalation device according to claim 10, further comprising a cover on the body.

18. The inhalation device according to claim 10, comprising a recess in the periphery of the body.

19. The inhalation device according to claim 10, further comprising a cover pivotally mounted on the body.

20. The inhalation device according to claim 10, comprising a winding wheel.

21. The inhalation device according to claim 1, comprising an outlet open to at least the first pocket and the second pocket.

22. The inhalation device according to claim 1, wherein the first and second active pharmaceutical ingredients are selected from the group consisting of aclidinium bromide, formoterol fumarate, umeclidinium and pharmaceutically acceptable salts thereof, vilanterol trifenatate, indacaterol maleate, and glycopyrrolate bromide or any combinations thereof.

23. The inhalation device according to claim 1, wherein the each of the first and second active pharmaceutical ingredients is selected from the group consisting of umeclidinium and vilanterol trifenatate, and combinations thereof.

24. The inhalation device according to claim 1, wherein the each of the first and second active pharmaceutical ingredients is selected from the group consisting of fluticasone furoate and vilanterol trifenatate, and combinations thereof.

25. The inhalation device according to claim 1, wherein the each of the first and second active pharmaceutical ingredients is selected from the group consisting of fluticasone furoate, umeclidinium and vilanterol trifenatate, and combinations thereof.

26. The inhalation device according to claim 5, wherein each of the first and second active pharmaceutical ingredients is selected from the group consisting of umeclidinium and vilanterol trifenatate, and combinations thereof.

27. The inhalation device according to claim 5, wherein each of the first and second active pharmaceutical ingredients is selected from the group consisting of fluticasone furoate and vilanterol trifenatate, and combinations thereof.

28. The inhalation device according to claim 5, wherein each of the first and second active pharmaceutical ingredients is selected from the group consisting of fluticasone furoate, umeclidinium and vilanterol trifenatate, and combinations thereof.

29. The inhalation device according to claim 6, wherein each of the first and second active pharmaceutical ingredients is selected from the group consisting of umeclidinium and vilanterol trifenatate, and combinations thereof.

30. The inhalation device according to claim 6, wherein each of the first and second active pharmaceutical ingredients is selected from the group consisting of fluticasone furoate and vilanterol trifenatate, and combinations thereof.

31. The inhalation device according to claim 6, wherein each of the first and second active pharmaceutical ingredients is selected from the group consisting of fluticasone furoate, umeclidinium and vilanterol trifenatate, and combinations thereof.

32. The inhalation device according to claim 8, wherein each of the first and second active pharmaceutical ingredients is selected from the group consisting of umeclidinium and vilanterol trifenatate, and combinations thereof.

33. The inhalation device according to claim 8, wherein each of the first and second active pharmaceutical ingredients is selected from the group consisting of fluticasone furoate and vilanterol trifenatate, and combinations thereof.

34. The inhalation device according to claim 8, wherein each of the first and second active pharmaceutical ingredients is selected from the group consisting of fluticasone furoate, umeclidinium and vilanterol trifenatate, and combinations thereof.
